(19) United States
(12) Patent Application Publication
(10) Pub. No.: US 2022/0257302 A1
(43) Pub. Date: Aug. 18, 2022

(54) ELECTRODE ARRANGEMENT

(71) Applicant: Erbe Elektromedizin GmbH, Tuebingen (DE)

(72) Inventors: Tjark Brandt, Tuebingen (DE); Uwe Schnitzler, Tuebingen (DE); Johannes Heym, Tuebingen (DE); Martin Walz, Tuebingen (DE)

(73) Assignee: Erbe Elektromedizin GmbH, Tuebingen (DE)

(21) Appl. No.: 17/628,666

(22) PCT Filed: Jul. 21, 2020
(86) PCT No.: PCT/EP2020/070580

§ 371 Date: Jan. 20, 2022

(30) Foreign Application Priority Data
Jul. 23, 2019 (EP) 19187863.6

(51) Int. Cl. A61B 18/04; A61B 18/14

(52) U.S. Cl. CPC A61B 18/042; A61B 2018/00029; A61B 18/1482

(57) ABSTRACT

An electrode for an argon plasma surgical instrument. The electrode has a heat dissipation device in the form of a single- or multi-layer coating. The coating may have a greater electrical conductivity and also a greater thermal conductivity than that of the material of the electrode main body. The coating may also have a melting temperature less than that of the material of the electrode main body. The melting temperature of the coating may be below 1100° C. If the coating is multi-layered, the melting temperature of the outer surface layer may also be below 1100° C., and preferably below 1000° C.

The invention refers to an electrode arrangement for an electrosurgical instrument, particularly for an instrument for plasma coagulation of biological tissue.

Instruments for tissue coagulation are known from different documents as well as from practical experience. For this it is referred to DE 10 2011 116 678 A1 as well as DE 699 28 370 T2. Both documents disclose instruments having electrodes that are, for example, configured in a ring-shaped manner and can consist of a suitable material that is characterized by its heat resistance, such as among others also tungsten.

Further a plasma coagulation instrument having a hose-like instrument body, inside the lumen of which a hexagonal electrode consisting of metal is arranged, is known from DE 100 30 111 A1. It is connected with an electrical supply line in order to be able to supply the electrode arranged at the distal end of the hose with RF voltage. An electrical discharge origins from the tip configured at the distal end of the plate-shaped electrode, whereby a plasma stream, particularly a noble gas plasma stream, can be created. The gas stream flowing around the electrode concurrently serves to dissipate heat from the electrode, whereby excessive heating thereof shall be avoided. Due to the heat dissipation, in addition a minimization of the burn-off behavior at the discharge section of the electrode shall be achieved and in so doing, the lifetime of the instrument shall be increased.

An efficient cooling of the electrode platelet by the gas stream, however, requires a high gas flow, which is not always desired.

Furthermore, it is known from WO 2005/046495 A1 to use an ignition electrode from tungsten wire, the end of which is located at a distal end of an otherwise hose- or tube-shaped instrument body. The tungsten wire arranged in the lumen of this body is held in a certain distance to its distal end by means of a platelet on which it is attached and that serves for its cooling. However, the heat dissipation from the tungsten wire is impeded by the transition location between the platelet and the tungsten wire.

Due to melting of the electrodes, particles, particularly metal particles, can get into the spark and/or the plasma stream and thus finally into living tissue, which is increasingly disliked.

It is therefore the object of the invention to provide a concept, which can reduce the material discharge of an electrosurgical, particularly plasma surgical instrument, uring use.

This object is solved with an electrode arrangement according to claim 1 and in an instrument according to claim 14:

The electrode arrangement according to the invention comprises an electrode on which a tip is formed orientated in distal direction. The electrode cross-section increases in proximal direction away from the tip continuously or in at least one step. The electrode consists of a material combination, the heat conductivity of which is preferably larger than 20 W/(m*K), whereby thus a very highly reduced electrode burn-off can be achieved. The electrode is provided with a heat conductive overlay, whereby the lifetime of the electrode and also the lifetime of an instrument equipped with the electrode is increased. Independent from the thermal conductivity of the electrode, the heat conductive overlay results in an increase of the lifetime of the electrode compared with the same electrode without overlay. The overlay extends in distal direction, preferably up to the proximity of the distal end of the electrode or covers the distal end thereof.

The electrode cross-section can increase starting from the distal tip in a step-like manner or also continuously until the electrode gets in contact with the wall of the lumen in which it is arranged. If the cross-section increase from the tip to a proximal part of the electrode is stepwise, one or multiple steps can be provided for this purpose. The tip of the electrode (and the flank area of the electrode adjoining the tip) is the location from which a spark or a plasma stream originates typically. The tip and the part of the electrode directly adjoining thereto thus form the area in which the discharge root point of the electrical discharge is located. At this root point a current concentration occurs that concurrently forms a heat source. At the discharge root point the electrode can be bare, i.e. the overlay is not provided or has been removed during operation. By means of both measures, namely the cross-section increase of the electrode in proximal direction and the use of a material combination for the electrode, the thermal conductivity of which is preferably larger than 20 W/(m*K) heat created at the electrode root point is largely more effectively dissipated than it has been the case previously by use of electrodes of equal configuration made of stainless steel or chromium nickel steel. The inventive electrode is particularly characterized in that the thermal conductivity thereof from the tip measured in proximal direction and/or measured transverse to the proximal direction is larger than the thermal conductivity of stainless steel.

Preferably the electrode comprises a section extending at least 2.5 mm starting from the tip in proximal direction, the thermal capacity of which is less than 4.17 mJ/K. This contributes to quick local small scale (e.g. limited to few square millimeters) heating of the electrode tip and to fixing the discharge root point in this area. The coating can melt in sections in this manner, e.g. in small regions close to the distal tip.

The combination of an electrode geometry in which the electrode cross-section increases away from the tip in proximal direction, whereby the electrode is made of a high thermal conductive material combination, allows the use of a tip with particularly small radius of curvature that can be particularly smaller than 1/10 of the maximum transverse dimension of the electrode. In doing so, a high field strength can be achieved at the electrode tip that can lead to the formation of a spark and a plasma, also in case of low RF voltages and RF currents. Then the electrode is especially ignitable.

Preferably the electrode is configured in a plate-shaped manner, wherein the increase of the electrode cross-section is achieved by an increasing transverse dimension of the electrode along the axial direction in proximal direction away from the tip. The transverse dimension can increase continuously, whereby a particularly good heat dissipation is achieved. A continuous cross-section and transverse dimension increase can be achieved in that steplessly configured edges adjoin the tip of the electrode.

The electrode can be configured as platelet that comprises two flat sides that are connected with one another by means of narrow sides. Two edges extending toward each other may join at the tip that forms the distal end of the electrode. Between the narrow sides and the flat sides, edges can be formed. Such an electrode can be provided as cut sheet metal, for example.

The electrode can also be configured as wire electrode, thus as thin rod, the distal end of which forms the tip. The wire (needle, thin rod) can be connected with a holding part that diametrically extends through the lumen of a probe and is part of the electrode.

According to the invention, the electrode consists of a material combination that is formed in that the electrode consists of a base body that comprises at least one surface on which a heat dissipation device is attached. The heat dissipation device thereby extends in distal direction, preferably up to the tip of the electrode, at least up to an area that is occupied by the discharge root point during operation. Thus, the heat created there can be directly transferred to the heat dissipation device without requiring heat transfer from the electrode to the heat dissipation device. In other words, the heat dissipation device is in direct contact with the heat source, here in form of the discharge root point. In proximal direction the heat dissipation device extends preferably at least up to an area of the electrode in which it comprises its maximum transverse dimension.

In the simplest case the electrode consists of a base material on which a thermally conductive overlay is applied as heat dissipation device, e.g. in form of a thermally conductive coating. This layer extends preferably up to the tip of the electrode and over large sections of the flat sides or over the entire flat sides of the electrode. The coating can also extend over the narrow sides of the electrode. For example, if the electrode is made of stainless steel or another less good heat-conductive material, the heat dissipation device is made of particularly good heat-conductive material, such as silver, diamond-like carbon (DLC) or the like. However, preferably the heat dissipation device is made of a metallic material that is also electrically conductive, such that the heat dissipation device, e.g. in form of the heat dissipating layer, contributes to the current conduction and can get into direct contact with the discharge root point. The heat dissipation device can also consist of a good thermally conductive ceramic material, e.g. AlN (aluminum nitride). The ceramic material can be configured in an electrically conductive or an electrically insulating manner. Preferably the heat dissipation device, that is e.g. configured as heat-conductive coating, is also particularly good electrically conductive. It is particularly advantageous, if the electrical conductivity of the coating is larger than the electrical conductivity of the base material. Preferably the layer is at least partly made of silver. It can be pure silver or a silver alloy or a multi-layer configuration in which at least one layer, preferably the layer provided at the surface, consists of silver or a silver alloy.

The layer configuration can comprise an adhesion influencing layer, e.g. an intermediate layer arranged between the surface proximate layer and the base material. The intermediate layer can be particularly a layer that influences the adhesion of the surface proximate layer on the base material. Particularly the intermediate layer can be an adhesive layer that facilitates the coating of the base material (stainless steel) with the coating material (silver). The adhesive layer can support the redistribution of coating material away from the distal tip during operation.

Preferably the melting temperature of the surface material is lower than the melting temperature of the base material. If an intermediate layer is present, the melting temperature of the intermediate layer is lower than the melting temperature of the base material and higher than or equal to the melting temperature of the material of the surface layer. The melting temperature of the intermediate layer can, however, also be lower than the melting temperature of the coating material.

Further preferably the thermal conductivity of the material of the surface layer is higher than the thermal conductivity of the base material. If an intermediate layer is present, the thermal conductivity of the material of the intermediate layer is preferably higher than the thermal conductivity of the base material and lower than, higher than or equal to the thermal conductivity of the material of the surface layer.

In a particularly preferred embodiment the heat dissipation device comprises thus an electrical conductivity and particularly also a thermal conductivity that is respectively larger than the electrical and thermal conductivity than the material of the base body.

As base material, particularly alloys are suitable that contain ion and/or chromium and/or nickel. In addition, as additional alloy components carbon and/or manganese and/or phosphor and/or sulphur and/or silicon and/or nickel and/or nitrogen and/or molybdenum can be present. A stainless steel preferred as base material has the following composition:

| | Fe | C | Cr | Mn | P | S | Si | Ni | N | Mo |
|---|---|---|---|---|---|---|---|---|---|---|
| min | | 0.05 | 16.0 | | | | | 6.0 | | |
| max | 47.605 | 0.15 | 19.0 | 2.0 | 0.045 | 0.15 | 2.0 | 9.5 | 0.11 | 0.8 |

As material for the intermediate layer gold or nickel or alloys thereof are particularly suitable.

In the drawings embodiments of the invention are illustrated. The drawings show:

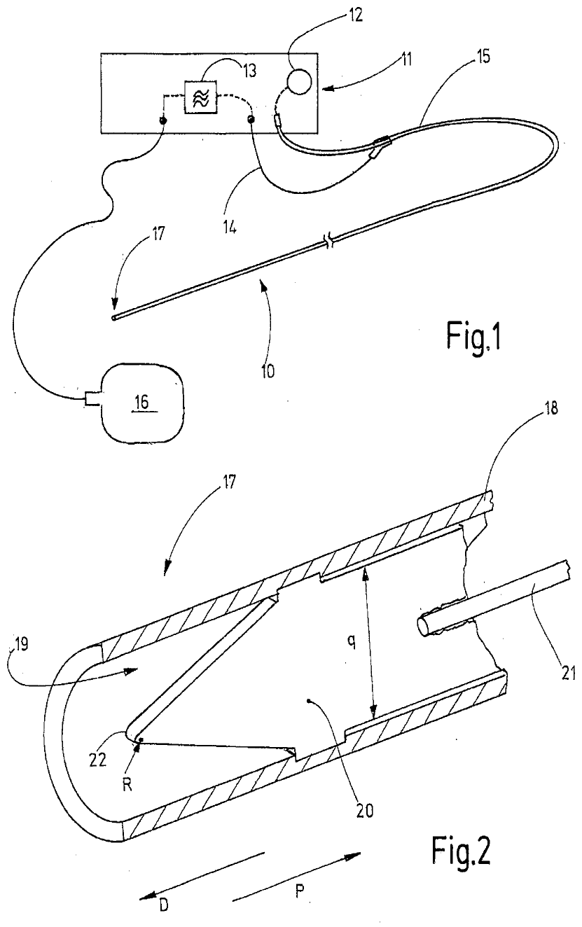

FIG. 1 an inventive instrument, the assigned supplying apparatus and a neutral electrode in very schematic partly perspective illustration,

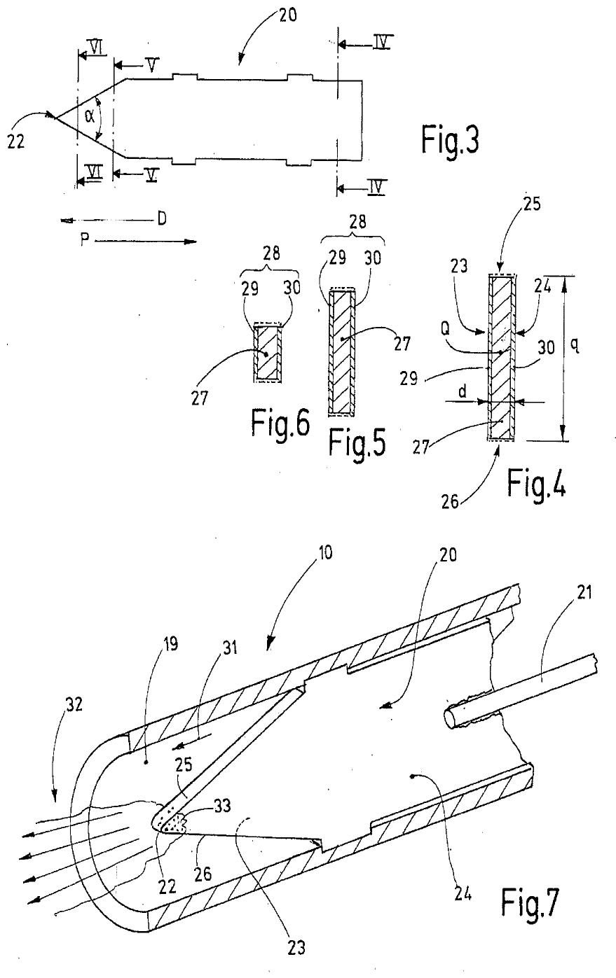

FIG. 2 the distal end of the instrument according to FIG. 1 in perspective schematic longitudinal-section illustration,

FIG. 3 the electrode of the instrument according to FIG. 2 in side view,

FIGS. 4, 5 and 6 different cross-sections of the electrode according to FIG. 3,

FIG. 7 the instrument according to FIGS. 2-6 in a sectional perspective illustration during operation,

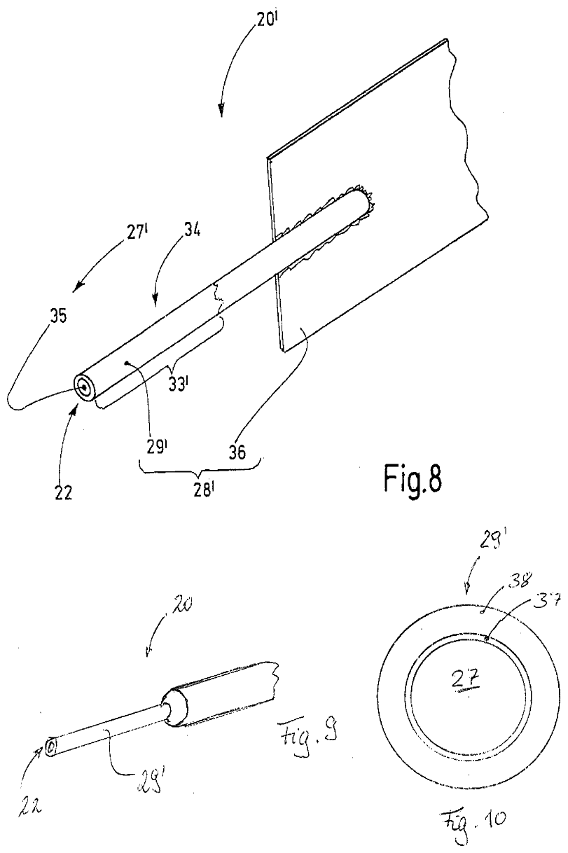

FIG. 8 a modified embodiment of an electrode for the instrument according to FIG. 2,

FIGS. 9 and 10 a further embodiment of an electrode for an instrument according to FIG. 2.

In FIG. 1 an instrument 10 is illustrated that serves for plasma-based tissue treatment. The tissue treatment can comprise ablation, coagulation, cutting or other types of treatment.

The instrument is connected to an apparatus 11 that contains a gas source 12, e.g. an argon source, as well as a generator 13 for electrical supply of the instrument 10. It is connected via respective connection means with a line 14 that leads to the instrument 10 and that is introduced in a line 15 via which instrument 10 is supplied with gas. The generator 13 is in addition connected via respective connection means with a neutral electrode 16 that is to be attached to a patient prior to the use of instrument 10. The following description, however, also applies for instruments with other neutral electrode configuration.

The instrument 10 comprises a distal end 17 that is separately illustrated in FIG. 2. As apparent, a tube or hose 18 is part of instrument 10 that surrounds a lumen 19 that is open at the distal end 17 of hose 18. In the area of the distal end 17 hose 18 can be provided with an inner or outer reinforcement, e.g. in form of a ceramic sleeve, which is not further illustrated in FIG. 2. The hose 18 can thus be configured with one or multiple layers. Examples for instruments with a ceramic sleeve inserted in the open end of hose 18 can be taken from WO 2005/046495 A1.

An electrode 20 is arranged in lumen 19 that is electrically connected with a wire 21 extending through lumen 19 and being part of line 14. The wire 21 can be welded to electrode 20 or can also be connected mechanically, e.g. by crimping.

Electrode 20 preferably comprises the basic shape illustrated in FIG. 3. At its distal end a sharp or at most slightly rounded tip 22 is configured on the electrode 20, the radius of curvature R of which (FIG. 2) is as small as possible and preferably smaller than one tenth of the transverse dimension q that is to be measured transverse to the axial direction and largely corresponds to the inner diameter of lumen 19. Electrode 20 is preferably configured in a plate-shaped manner, i.e. its thickness is remarkably smaller than its transverse dimension q. This is, for example, apparent from FIG. 4 that shows a cross-section of electrode 20 at the chain-dotted cutting line IV-IV in FIG. 3. The thickness d is smaller than ⅕, preferably smaller than 1/10 of the transverse dimension q.

As further apparent from FIG. 4, electrode 20 comprises two flat sides 23, 24 that are connected by means of narrow sides 25, 26. Thus, in total a quadrangular, preferably rectangular cross-section Q is obtained that is bordered by the flat sides 23, 24 and narrow sides 25, 26. The quadrangular cross-section can also be bent one time or multiple times, e.g. S-shaped.

Electrode 20 comprises a tapering section at its distal end in which the narrow sides 25, 26—extending parallel to one another apart therefrom—are convergingly arranged toward the tip 22. The convergingly toward one another extending sections of the narrow sides 25, 26 can be configured in a straight manner, as shown in FIG. 3, or also in a convex or concave manner. They define an angle α between each other that is preferably in the range of 20° to 100°.

As the cross-sections V-V and VI-VI show, that are separately illustrated in FIGS. 5 and 6, the cross-section of electrode 20 decreases toward tip 22 in distal direction D or in other words increases in proximal direction P. Thereby the thickness d of electrode 20 can be constant in the tapering section toward tip 22, as shown in FIGS. 5 and 6. The thickness d can, however, also decrease toward tip 22. However, in any case the transverse dimension q decreases in the tapering section toward tip 22.

In a preferred embodiment of the invention electrode 20 comprises a multiple layer configuration, as apparent from FIGS. 4, 5 and 6. For this electrode 20 comprises an electrode base body 27 that is connected with a heat dissipation device 28 at least on its flat sides 23, 24, however as an option also on its narrow sides 25, 26. The heat dissipation device 28 consists in the present embodiment of a two-dimensional coating of the flat sides of the electrode base body 27 with thermally conductive overlays 29, 30. In the embodiment the base body 27 can consist of stainless steel, whereas the overlays 29, 30 consist of another material having a better thermal conductivity and/or a better electrical conductivity. Silver has shown to be particularly suitable for this purpose. Possible other overlays consist of aluminum and/or copper and/or hard metal and/or DLC and/or tungsten and/or a layer, e.g. metal layer with CBN (cubic boron nitride), diamond powder or similarly well thermally conductive material embedded therein.

The instrument described so far operates as follows:

As illustrated in FIG. 7, a gas stream 31 originating from the gas source 12 flows through lumen 19 of instrument 10 during operation. This gas stream (preferably argon stream) flows along both flat sides 23, 24 of electrode 20. Concurrently electrode 20 is supplied with radio frequency electrical current via wire 21. The working frequency of generator 13 and thus the frequency of the current is thereby preferably above 100 kHz, preferably above 300 kHz, further preferably above 500 kHz. At the tip 22 and an adjoining area thereof the current exits electrode 20 and forms a spark igniting toward the not further illustrated biological tissue of the patient or a plasma 32 flowing thereto. The root point 33 of the spark or plasma thereby touches the narrow sides 25, 26, however, particularly the flat sides 23, 24 of electrode 20, whereby this root point area 33 occupies, for example, less than 1/10 of the axial length (to be measured in proximal direction) of the area of electrode 20 in which the narrow sides 25, 26 diverge away from tip 22. The overlays 29, 30 can extend into this area and preferably up to the tip 22. Thus, the spark or plasma stream is electrically directly supplied by overlay 29, 30. The thickness of overlays 29, 30 can be relatively small. It has shown that already coatings being 10 to 20 μm thick result in a substantial extension of the lifetime of electrode 20 and in a substantially reduced material removal and in a highly reduced heat radiation therefrom. Preferably the thickness of the overlays—consisting e.g. of silver—has an amount of 20 μm, 30 μm or 50 μm. Preferably the overlay has a thermal conductivity of more than 400 W/(m*K). For example, the thickness of the electrode can be 0.1 mm. Also the thermal conductivity of the entire electrode preferably exceeds 400 W/(m*K). The electrode 20 consisting of the material combination stainless steel/silver thereby comprises a phenomenal lifetime.

In a modified embodiment it is also possible to let the electrode cross-section increase in proximal direction not continuously, different to the embodiments described above, but in a step-like manner, i.e. in one or multiple steps. Such an embodiment is illustrated in FIG. 8. However, this embodiment also realizes the inventive concept, which is why for the description of this electrode 20′ it is referred to the embodiment according to FIGS. 1-7. The already introduced reference numerals are used in the following, whereby they are provided with an apostrophe for the purpose of distinction. The description above accordingly applies apart from the following particularities for the embodiment according to FIG. 8.

The electrode 20′ comprises a tip 22′ that can be formed here by the pointed or blunt end of a wire-shaped straight or corrugated electrode section. This wire-shaped electrode section 34 comprises a core 35 that forms the base body 27′ and for its part can be configured as thin cylinder pin. The diameter of the wire-shaped electrode section is preferably smaller than 0.5 mm and has an amount of, e.g. 0.3 mm. The core 35 is provided with an overlay 29′ that here—as appropriate in connection with an electrode holding platelet 36—forms the heat dissipation device 28′. The electrode section 34 can be welded, crimped or otherwise connected with the electrode holding platelet 36. A substance bond connection is preferred, because of the better heat transfer. The electrode holding section 36 can consist of stainless steel or another material that is provided with a thermally conductive coating, such as tungsten, copper, aluminum, DLC or the like or that is configured of thermally conductive material, such as tungsten, copper, aluminum, DLC or the like. At the transition location from the wire-shaped electrode section to the electrode holding platelet 36, the cross-section of the electrode increases in a stepwise manner.

The electrode 20 can also be configured according to FIGS. 9 and 10 and can comprise in the cross-section circular-shaped sections with axial distance having different diameters.

In all electrodes 20, 20′ it applies independent from the geometric shape and independent therefrom whether the electrode cross-section increases in proximal direction continuously or in steps or whether it remains constant or decreases locally that the overlay 29, 29′, 30 remarkably increases the lifetime of electrode 20, 20′ and instrument 10. Thereby it is particularly advantageous, if the overlay 29, 29′, 30 extends starting from tip 22 at least approximately 5 mm to 10 mm or also approximately 10 to 20 mm in proximal direction. Preferably the overlay 29, 29′, 30 consists of a metal, e.g. silver, the melting temperature T_{U} is lower than the melting temperature T_{G} of the base material, e.g. stainless steel. Also the overlay 29, 29′, 30 preferably comprises a thermal conductivity λᵤ that is higher than the thermal conductivity λ_{G} of the base material.

Moreover, the overlay 29, 29′, 30 can consist of an adhesion influencing intermediate layer 37 arranged in direct contact to the base body 27 and a surface layer 38 arranged on the intermediate layer 37. The surface layer 38 consists preferably of a metal, the melting temperature T_{O} is lower than or is approximately as high as the melting temperature T_{Z} of the intermediate layer that in turn is, however, lower than the melting temperature T_{G} of the material of the electrode base body 27. In addition the surface layer 38 consists preferably of a material, the thermal conductivity λ_{O} is minimum as high as the thermal conductivity λ_{Z} of the intermediate layer 37. The thermal conductivity λ_{Z} of the intermediate layer 37 is preferably higher than the thermal conductivity λ_{G} of the material of the electrode base body 27.

In all of the electrodes 20, 20′ described previously it also applies that the cross-section surface Au of the overlay 29, 29′, 30 comprises at least at the tip 22 (up to approximately 2.5 mm in proximal direction) at least 10-12% of the cross-section area A_{G} of the electrode base body. In addition the electrode 20, 20′ comprises a section originating from its tip 22 extending in proximal direction along at least 2.5 mm, the thermal capacity of which is lower than 4.17 mJ/K. The electrode 20, 20′ comprises a volume V_{E} that is preferably in a defined ratio to the area of the surface A_{UO} of the overlay 29, 29′, 30. Preferably the ratio of the surface area A_{UO} to the volume V_{E} is larger than 2.24 mm⁻¹.

In operation of instrument 10 with an electrode 20′ according to FIG. 8, 9 or 10 an electrical discharge and thus the forming spark or plasma stream origins first from tip 22 and then from at least a part of the wire-shaped electrode section 34. The electrically and thermally conductive coating 29′, that is preferably a silver coating, remarkably reduces the electrical resistance of electrode 20 or 20′. The radio frequency alternating current of generator 13 concentrates in the outer layers of electrode 20, 20′ and in this manner flows substantially through coatings 29, 30, 29′. Thereby ohmic losses at the electrode 20, 20′ are minimized and in addition the reduced amount of heat is dissipated substantially better away from the discharge root by the coating and is distributed such that it can be transferred to the gas stream extensively. The surface layer 38 and potentially also the intermediate layer 37 can melt and can retract slightly from the tip 22 in proximal direction. The discharge root point 33 remains stationary at the tip 22 (and the section directly adjoining thereto, approximately 2.5 mm). This mitigates the thermal stress of electrode 20, 20′ as well as instrument 10.

In an improved instrument 10 the electrode 20, 20′ is provided with a heat dissipation device 28, 28′ in the form of a single layer or multiple layer overlay 29, 29′, 30. It comprises preferably a higher electrical conductivity, as well as a higher thermal conductivity compared with the material of the electrode base body 27, 27′. In addition, it preferably comprises a lower melting temperature than the material of the electrode base body 27. The melting temperature T_{U} of the overlay is preferably below 1100° C. If the overlay 29, 29′, 30 is multi-layered, the melting temperature T_{O} of the outer surface layer 38 is preferably also below 1100° C., further preferably below 1000° C.

### REFERENCE SIGNS

10 instrument
11 apparatus
12 gas source
13 generator
14 line (for current)
15 line (for gas)
16 neutral electrode
17 distal end of instrument 10
18 hose
19 lumen
20 electrode
21 wire
22 tip
q transverse dimension of electrode 20
d thickness of electrode 20
23, 24 flat sides of electrode 20
25, 26 narrow sides of electrode 20
Q cross-section of electrode 20
a angle between parts of narrow sides 25, 26
D distal direction
P proximal direction
λ thermal conductivity
λ_{U} thermal conductivity of overlay 29, 29′, 30
λ_{Z} thermal conductivity of intermediate layer 37
λ_{G} thermal conductivity of base body 27
λ_{O} thermal conductivity of surface layer 38
27 electrode base body
28 heat dissipation device
29, 29′, 30 overlays
31 gas stream
32 plasma
33 root point
34 wire-shaped electrode section
35 core
36 electrode holding section
37 intermediate layer
38 surface layer
T_{U} melting temperature of overlay 29, 29′, 30
T_{G} melting temperature of electrode base body 27
T_{O} melting temperature of surface layer 38
T_{Z} melting temperature of intermediate layer 37

1. An electrode for plasma coagulation electrosurgical instrument, the electrode comprising:
a tip orientated in distal direction,
wherein the electrode has a cross-section that increases in a proximal direction from the tip, the increase in cross-section being continuous or in at least one step,
wherein the electrode comprises a material combination, the thermal conductivity of which is greater than 20 W/(m*K), and
wherein the electrode further comprises an electrode base body having a thermally conductive overlay.

2. The electrode according to claim 1, wherein the electrode has a maximum transverse dimension and a radius of curvature at its tip that is less than one tenth of the maximum transverse dimension.

3. The electrode according to claim 1, wherein the electrode is configured in a wire-shaped manner or as platelet that comprises two flat sides that are connected with one another by means of narrow sides.

4. The electrode according to claim 3, wherein the electrode comprises at least one flat side and wherein the thermally conductive overlay comprises a layer configured to cover the entire flat side.

5. The electrode according to claim 1, wherein the overlay comprises a thermally conductive material, and wherein the overlay comprises a thermal and/or electrical conductivity that is respectively greater than the thermal and/or electrical conductivity of the base body.

6. The electrode according to claim 1, wherein the overlay extends up to a section configured for direct contact with a spark originating from the electrode.

7. The electrode according to claim 1, wherein the overlay consists of a metal or a metal alloy, the melting temperature of which is less than the melting temperature of the material of the base body.

8. The electrode according to claim 1, wherein the overlay comprises an intermediate layer that is in direct contact with the electrode base body and a surface layer, the surface layer being arranged on the intermediate layer.

9. The electrode according to claim 8, wherein the surface layer comprises a metal or a metal alloy having a melting temperature T_{O} less than the melting temperature of the intermediate layer that in turn is less than the melting temperature of the material of the electrode base body.

10. The electrode according to claim 8, wherein the surface layer comprises a metal or a metal alloy having a thermal conductivity greater than the thermal conductivity of the intermediate layer that in turn is greater than the thermal conductivity of the material of the electrode base body.

11. The electrode according to claim 1, wherein the overlay comprises a cross-section area and the electrode base body comprises a cross-section area, and wherein the ratio between the cross-section area of the overlay to the cross-section area of the electrode base body is greater than 0.12.

12. The electrode according to claim 1, wherein the electrode further comprises a section extending at least 2.5 mm from the tip in proximal direction, the thermal capacity of the section being less than than 4.17 mJ/K.

13. The electrode according to claim 1, wherein the electrode comprises a volume and the overlay comprises a surface area, and wherein the ratio of the surface area of the overlay to the volume is greater than 2.24 mm⁻¹.

14. An instrument having an electrode according to claim 1.

15. The instrument according to claim 14, further comprising a tube or hose surrounding a lumen that is open at a distal end of the tube or hose and configured to connect to an argon gas source, wherein the electrode is arranged in the lumen and configured to connect to a generator.

16. The electrode according to claim 5, wherein the thermally conductive material comprises a metal or a metal alloy.
